(19) United States
(12) Patent Application Publication
(10) Pub. No.: US 2006/0183685 A1
(43) Pub. Date: Aug. 17, 2006

(54) PEPTIDE PHARMACEUTICAL FORMULATIONS

(76) Inventors: Barton Holmquist, Lincoln, NE (US); Daniel C. Dormady, Omaha, NE (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/379,893

(22) Filed: Apr. 24, 2006

Related U.S. Application Data
(62) Division of application No. 09/858,880, filed on May 17, 2001.
(60) Provisional application No. 60/205,377, filed on May 17, 2000.
(60) Provisional application No. 60/205,262, filed on May 19, 2000.

(51) Int. Cl. A61K 38/18; A61K 31/19

(52) U.S. Cl. 514/12; 514/553; 514/557

(57) ABSTRACT

A pharmaceutical composition for administration to a mammal is disclosed. The composition includes a therapeutically effective amount of a peptide, such as a GLP-1 molecule, a PTH molecule, or a GRF molecule. The composition further includes a buffer including a weak acid having an acid dissociation constant value of greater than about 1×10⁻⁵, such as acetic acid. The composition also includes an excipient for making the composition generally isotonic, such as D-mannitol.

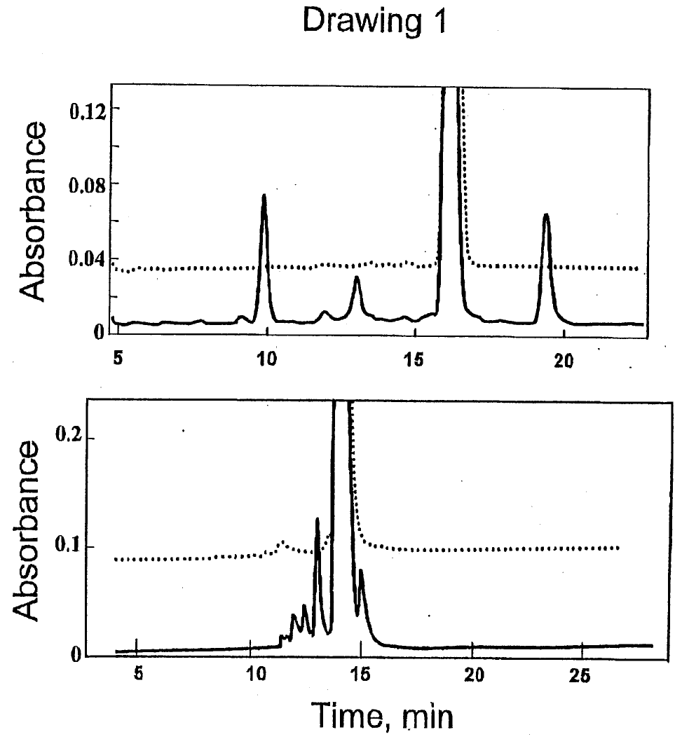

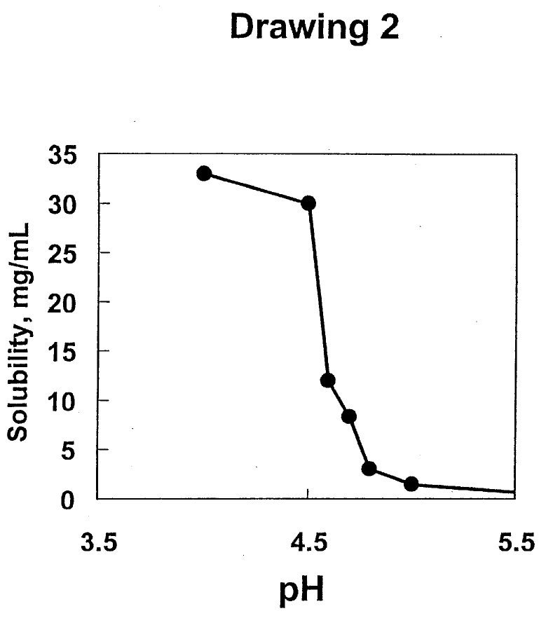

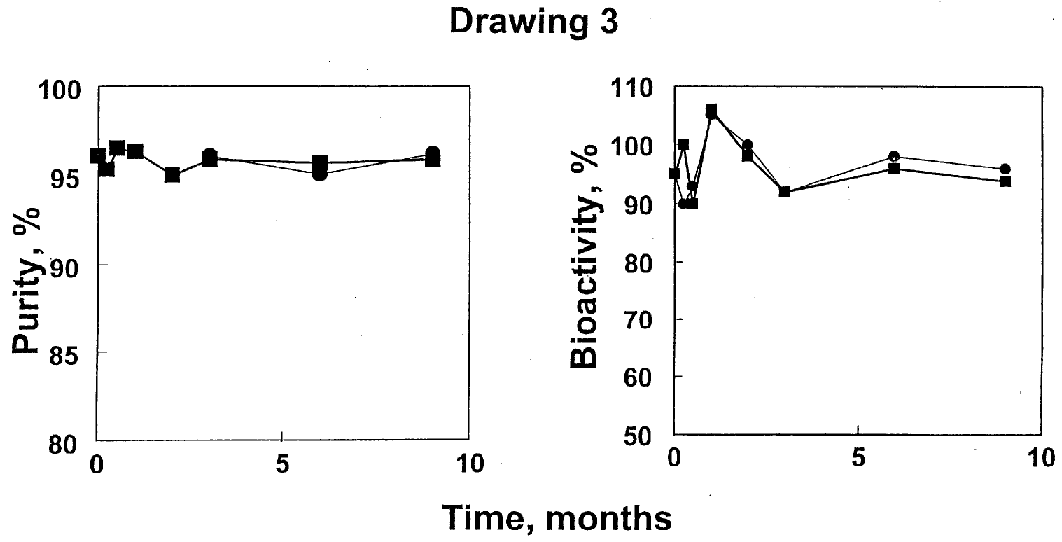

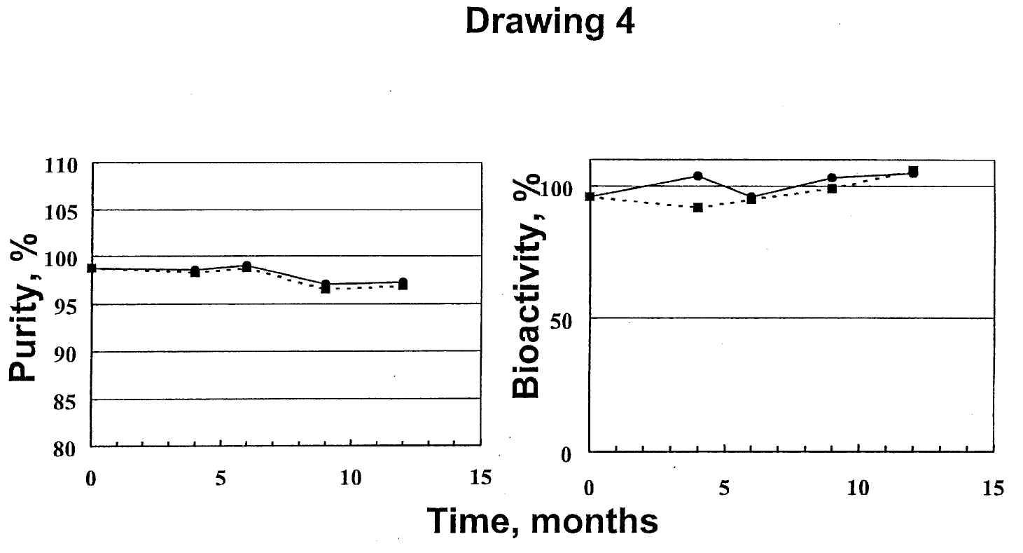

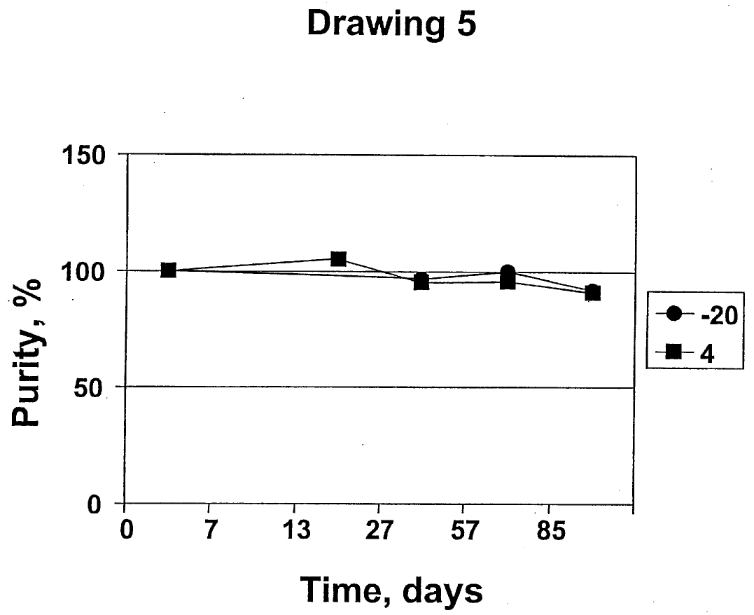

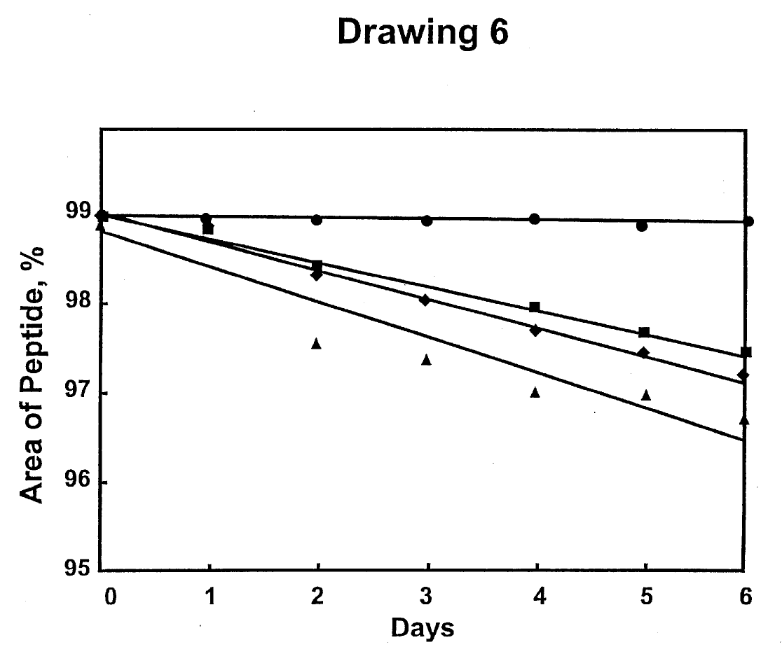

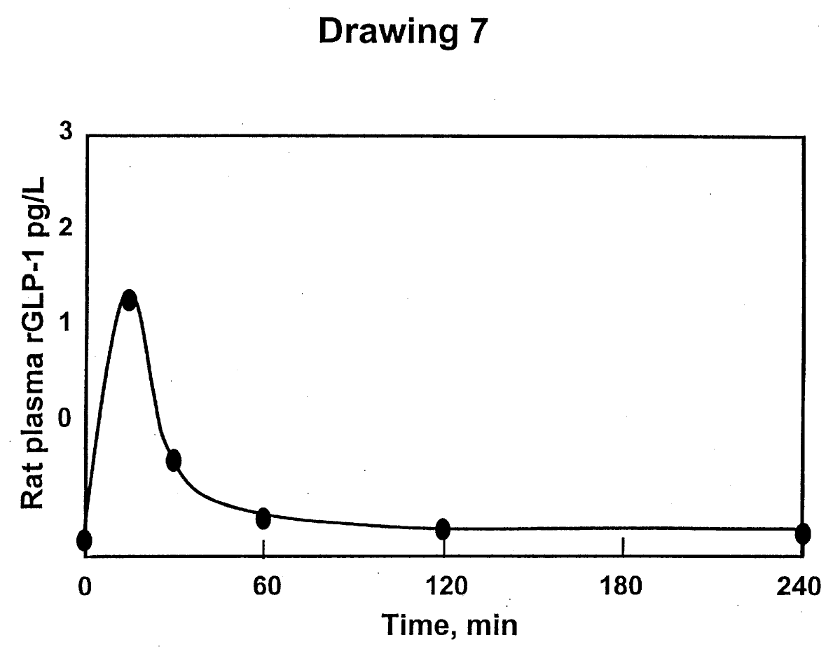

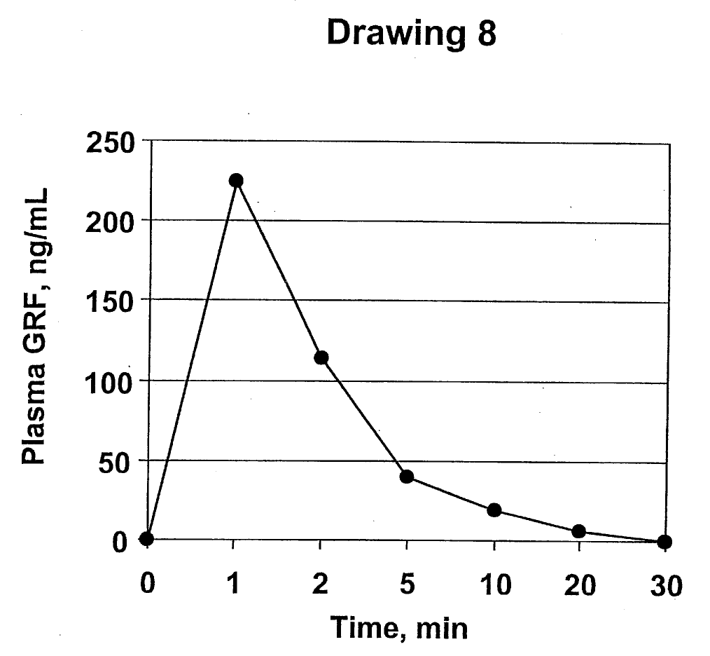

### RELATED APPLICATIONS

This application is a Divisional to U.S. application Ser. No. 09/858,880 which claims priority to U.S. Provisional Application Ser. No. 60/205,377, filed May 17, 2000 and U.S. Provisional Ser. No. 60/205,262, filed May 19, 2000, all of which are incorporated by reference.

### FIELD OF THE INVENTION

The present invention generally relates to pharmaceutical formulations for peptides. More specifically, the present invention relates to pharmaceutical formulations of a peptide, such as a glucagon-like peptide-1 (GLP-I), a parathyroid hormone (PTH) or a growth hormone releasing factor (GRF), or a pharmaceutically active derivative or analog of such peptides, an acidic buffer and mannitol. The novel formulations, for example, are well-tolerated by humans, and are, for example, surprisingly stable compositions; the soluble peptides do not dimerize or aggregate.

### BACKGROUND OF THE INVENTION

Peptides such as GLP-1, PTH, and GRF are known in the art to be useful for treating a variety of disorders. For example, GLP-1(7-36)amide is useful for treating type II diabetes (also known as Non-Insulin Dependent Diabetes Mellitus, NIDDM). PTH(1-34) is useful for treating osteoporosis, as is GRF(1-44)amide. See U.S. Pat. No. 4,870,054. A combination of PTH(1-34) and GRF(1-44)amide can also be used to treat osteoporosis. See U.S. Pat. No. 5,164,368.

There is a variety of art-recognized problems associated with formulating such peptides into pharmaceutically acceptable compositions. It is important to have a sufficiently high concentration of peptide that is soluble and that forms minimal peptide aggregates and peptide dimers. It is known in the art that the formation of such aggregates and dimers is a significant problem encountered in making pharmaceutical formulations from peptides such as GLP-1. For example, GLP-1 is known to gel and aggregate under numerous conditions, making it difficult to make stable soluble peptide formulations. See EP 0978565 A1.

A variety of pharmaceutical formulations comprising GLP-1, PTH and GRF have been described in the art. Such peptides have generally been administered by dissolving the peptide in water containing albumin or other adjuvants and injecting it into a human (Creutzfeldt et al, Diabetes 19, 1 (1996); Ahren et al, J. Clin. Endo. Metab. 82, 473 (1997)). This procedure has disadvantages because such peptides are not stable or sufficiently soluble under such conditions (near neutral pH values), and adjuvants, such as albumin, are unstable at acidic pH values.

Moreover, it is known in the art that it is desirable to use pharmaceutical formulations that are at physiological pH, to minimize adverse side effects and discomfort to patients. See Brazeau et al., J. Pharm. Sci., 87, 667 (1998). However, at physiological pH (about pH 7.4), the solubilities of GLP-1, PTH, and GRF are low. For example, the solubility of the peptide GLP-1 in water at a pH of about 7.4 is less than about 0.2 mg/mL. The solubility of GLP-1 in physiological saline is also low. The solubilities of PTH and GRF at physiological pH are higher, up to 4 mg/mL.

To increase peptide solubility at physiological pH, prior art formulations have used various art-recognized agents, such as detergents and solvents. The use of such agents is not desirable, however, because they can cause adverse side effects in patients. See Brazeau et al., J. Pharm. Sci. 87, 667 (1998). Also, human serum albumin has been used in GLP-1 formulations because of its buffering capabilities and to reduce adsorption of GLP-1 to the storage container or devices used for administration. GLP-1 is a hydrophobic peptide that adsorbs to hydrophobic surfaces that are found on, for example, tubing and syringes. However, it is not desirable to use human serum albumin because it can stimulate adverse immune reactions in a patient. Also, great care must be taken to use highly purified albumin, to minimize contaminants that can also cause unwanted side effects.

The stability of an amide bond generally is greatest at a pH in the range of about 4.0 to 4.5. However, such a pH range often cannot be used for formulations of therapeutic peptides. A low pH can result in denaturation of peptides that have tertiary or quaternary structure and/or can result in peptide inactivation. Moreover, low pH pharmaceutical formulations are known to cause discomfort to patients, upon injection. See Brazeau et al., J Pharm. Sci. 87, 667 (1998).

U.S. Pat. No. 5,705,483 describes a formulation of GLP-I that is combined with distilled water and adjusted to a pH of about 6.0 to 9.0. The '483 patent states that D-mannitol is an example of a suitable excipient for GLP-1. However, the high pH recited in the '483 patent formulation may contribute to the instability of GLP-1.

PCT Application WO 98/19698 describes a combination of 100 mmol GLP-1(7-36)amide and 0.025 mL human albumin solution (20%), with the pH adjusted to 4 using 5 M acetic acid. The volume of this formulation was brought to 1 mL using normal saline for administration to the abdomen of a human making the concentration of GLP-1 100 μM (about 0.3 mg/mL). However, as noted above, it is desirable to not use albumin in pharmaceutical formulations.

The 1999 Physician's Desk Reference (pp. 532-539) describes NEUPOGEN, commercially available from Amgen Inc., California. The PDR entry states that NEUPOGEN is the name of the drug product that is a formulation of filgrastim, a human granuloctye colony stimulating factor (G-CSF) produced by recombinant DNA technology, suitable for pharmaceutical use in stimulating white blood cell production. The PDR entry states that NEUPOGEN is formulated in a 10 mM sodium acetate buffer at pH 4.0, containing 5% sorbitol and 0.004% TWEEN 80. TWEEN 80 is an emulsifying, wetting, and dispersing agent (i.e., detergent), commercially available from Atlas Powder Company, Delaware. The PDR entry further states that the quantitative composition (per mL) of NEUPOGEN is: filgrastim 300 mcg., acetate 0.59 mg, sorbitol 50 mg, TWEEN 80 0.004%, sodium 0.035 mg, water for injection USP q.s. in 1.0 mL. G-CSF is a protein that is 175 amino acids long, and, as noted, the NEUPOGEN formulation contains detergent.

Accordingly, there is a need in the art for stable pharmaceutical formulations of relatively small peptides, such as GLP-1, PTH and GRF, that contain minimal levels of non-therapeutic adjuvants (such as albumin, detergents, and solvents) because this can cause adverse side effects. It would also be advantageous to provide effective stable pharmaceutical formulations that are well tolerated by humans, i.e., cause minimal patient discomfort. It further would be advantageous to provide peptide formulations having acceptable concentrations, that are soluble, and include minimal or no peptide dimers and/or aggregates. As noted, GLP-1 is known to gel and aggregate under numerous conditions, making stable formulation difficult. See EP 0978565 A1. Other advantages of the claimed invention will become apparent to those skilled in the art upon review of the specification and the appended claims.

### SUMMARY OF THE INVENTION

To provide stable peptide pharmaceutical formulations that are well tolerated by patients and that have minimal non-peptide components, the present inventors have developed pharmaceutical formulations comprising a peptide, a buffer, and a diluent. In particular, the present inventors have developed stable pharmaceutical compositions for administration to a mammal of peptides such as GLP-1(7-36)amide, PTH(1-34)OH, or GRF(1-44)amide, each prepared in acetic acid and D-mannitol.

It is therefore an object of the present invention to provide a stable unit dose of a pharmaceutical composition that provides for good stability of the peptide for administration to a mammal including a peptide, a buffer, and a diluent.

It is another object of the present invention to provide a method for treating an illness or disease in a mammal using a pharmaceutical composition that is well tolerated by the mammal for administration to the mammal including a peptide, a buffer and a diluent.

In accomplishing these and other objects, there has been provided in accordance with one aspect of the present invention a unit dose of a pharmaceutical composition for administration to a mammal. The composition includes a therapeutically effective amount of a peptide; the composition also includes a buffer comprising an acid having a pKa less than about 5, or less than 5. In particular, the inventive formulations comprise acetic acid. The formulations also include a diluent to make the composition isotonic. In particular, the inventive formulations comprise D-mannitol.

In a preferred embodiment, the composition consists essentially of a peptide, a buffer comprising an acid having a pKa less than about 5, or less than 5, and a diluent such as D-mannitol.

In another preferred embodiment, the composition consists of peptide, a buffer comprising an acid having a pKa less than about 5 or less than 5, and a diluent.

In one preferred embodiment, the inventive formulations comprise a peptide, acetic acid, and D-mannitol. In another preferred embodiment, the inventive formulations consist essentially of a peptide, acetic acid, and D-mannitol. In another preferred embodiment, the inventive formulations consist of a peptide, acetic acid, and D-mannitol.

All of these formulations preferably have a pH between about 3.0 and about 5.0 or between 3.0 and 5.0; more preferably, between about 4.0 and about 5.0 or between 4.0 and 5.0; more preferably between about 4.5 and about 5.0 or between 4.5 and 5.0; most preferably between about 4.5 and about 4.7 or between 4.5 and 4.7. Other preferred embodiments have a pH of 4.5, 4.6, or 4.7.

In accordance with another aspect of the present invention, a system for administering an effective amount of a pharmaceutical formulation to a mammal is disclosed. The system includes an infusion pump for administering a unit dose of a pharmaceutical formulation of the invention. The unit dose includes a therapeutically effective amount of a peptide having a molecular weight of between about 200 to 50,000 atomic mass units, including, for example, a GLP-1 molecule, a GRF molecule, or a PTH molecule.

In accordance with another aspect of the present invention, a method for the treatment of a disease in a mammal having the disease is disclosed. The method includes administering to the mammal an effective amount of a pharmaceutical composition of the invention.

Further objects include the following. A pharmaceutical composition comprising (1) a molecule selected from the group consisting of a GLP1 molecule, and GRF molecule, and a PTH molecule; (2) an acid having a dissociation constant value of greater than 1×10⁻⁵; and (3) an excipient, wherein the pH of the composition is between about 3.0 and 5.0. The above composition, wherein the acid comprises acetic acid. The above composition, wherein the excipient is D-mannitol. The above composition wherein the acid is acetic acid and the excipient is D-mannitol. The above composition, wherein the composition comprises GLP-1(7-36)amide. The above composition, wherein the composition comprises GRF(1-44)amide. The above composition, wherein the composition comprises PTH(1-34)OH. The above composition, wherein the composition is in unit dosage form. The above composition, wherein the composition is sterile. A system for administering a pharmaceutical composition comprising: an infusion pump for administering a unit dose of the above composition. The above system, wherein the composition is diluted up to about 40-fold with isotonic saline prior to administration. A method for the treatment of a disease or condition in a mammal comprising administering to the mammal a pharmaceutically effective amount of an above composition. The method above, wherein the disease or condition is selected from the group consisting of diabetes, excess appetite, obesity, stroke, ischemia, reperfusion injury, disturbed glucose metabolism, surgery, coma, shock, gastrointestinal disease, digestive hormone disease, atherosclerosis, vascular disease, gestational diabetes, liver disease, liver cirrhosis, glucorticoid excess, Cushings disease, the presence of activated counterregulatory hormones that occur after trauma or a disease, hypertriglyceridemia, chronic pancreatitis, the need for parenteral feeding, osteoporosis, and a catabolic state following surgery or injury. The above method, wherein the composition is administered to the mammal by a method selected from the group consisting of intravenous, subcutaneous, continuous, intermittent, parenteral, and combinations thereof. The above composition, wherein the composition has a pH of about 4.5. The above composition, wherein the composition has a pH of about 4.7. The above composition, wherein the composition has a pH of between about 4.5 and 4.7. The above composition, wherein the composition has a pH of 4.5. The above composition, wherein the composition has a pH of 4.7. The above composition, consisting essentially of acetic acid, D-mannitol, and a molecule selected from the group consisting of a GLP1 molecule, and GRF molecule, and a PTH molecule, wherein the composition is in liquid form. The above composition, consisting of acetic acid, D-mannitol, and a molecule selected from the group consisting of a GLP1 molecule, and GRF molecule and a PTH molecule, wherein the composition is in liquid form. The above composition, comprising acetate (about 10 mM) and D-mannitol (about 50.7 mg/mL). The above composition, consisting essentially of acetate (about 10 mM), D-mannitol (about 50.7 mg/mL), and a molecule selected from the group consisting of a GLP1 molecule, and GRF molecule, and a PTH molecule. The above composition, comprising acetate (about 10 mM), D-mannitol (about 50.7 mg/mL), and GLP-1(7-36)amide (about 1 mg/mL). The above composition, consisting essentially of acetate (about 10 mM), D-mannitol (about 50.7 mg/mL), and GLP-1(7-36)amide (about 1 mg/mL). The above composition, wherein the composition comprises acetate (about 10 mM), D-mannitol (about 50.7 mg/mL), and GRF(1-44)amide (about 4 mg/ml). The above composition, consisting essentially of acetate (about 10 mM), D-mannitol (about 50.7 mg/mL), and GRF(1-44)amide (about 4 mg/ml). The above composition, wherein the composition comprises acetate (about 10 mM), D-mannitol (about 50.7 mg/mL), and PTH(1-34)OH (about 50 mg/mL). The above composition, wherein the composition consists essentially of acetate (about 10 mM), D-mannitol (about 50.7 mg/mL), and PTH(1-34)OH (about 50 mg/mL). The above system, wherein the pump is programmed to release the molecule at a rate of about 10 or more μL per hour.

Further objects, features and advantages of the invention will be apparent from the following detailed description taken in conjunction with the accompanying drawings.

### BRIEF DESCRIPTION OF THE DRAWINGS

Drawing 1

Examples of the use of reverse phase HPLC for peptide purity analysis and illustrating the capacity to monitor the degradation of peptides. Samples were analyzed by reversed phase HPLC by elution with water/acetonitrile gradients in 0.1% trifluoroacetic acid. The HPLC system used was an HP 1100 chromatography system. Top Panel: GLP-1 stored at −20° C. (dotted line) and 50° C. (solid line) for one month in 10 mM acetic acid, 5.07% D-mannitol, adjusted to pH 4.5. Elution is with a gradient of from 33% to 95% acetonitrile in 22 min. with a Waters Symmetry Reverse Phase C18 column, 4.6×250 mm. Bottom panel: GRF stored at −20° C. (dotted line) and 37° C. (solid line) for one month in 10 mM acetic acid, 5.07% D-mannitol, adjusted to pH 4.7. The compositions of the HPLC buffers A and B were 20% and 50% (v/v) acetonitrile, respectively, and elution was with a gradient of from 25% to 55% B in 25 min. 5 using a Zorbax 5 micron, 4.6×250 mm column.

Drawing 2

Solubility of GLP-1 in 10 mM acetate buffer containing 5.07% D-mannitol as a function of pH at 25° C. Solutions were stirred with excess GLP-1 for four days. Following centrifugation, the amount of peptide in solution was determined by ultraviolet absorption spectrophotometry.

Drawing 3

Stability determined by HPLC (left panel) and bioactivity (right panel) of GRF as a function of storage time in the preferred formulation, 4 mg/mL GRF dissolved in 10 mM sodium acetate, 5.07% D-mannitol, adjusted to pH 4.7. Circles represent −20° C. and squares represent 4° C.

Drawing 4

Stability of GLP-1 in the preferred formulation (1 mg/mL GLP-1 dissolved in 10 mM sodium acetate, 5.07% D-mannitol, adjusted to pH 4.5), as determined by HPLC analysis (left panel) and bioassay (right panel). Circles represent −20° C. and squares represent 4° C.

Drawing 5

Stability of PTH (1 mg/mL PTH dissolved in 10 mM sodium acetate, 5.07% D-mannitol, adjusted to pH 4.7), as determined by HPLC analysis. Circles represent −20° C. and squares represent 4° C.

Drawing 6

Stability of GLP-1 by HPLC analysis of GLP-1 formulated in 10 mM sodium acetate, 5.07% D-mannitol at pH 4.5 at 1 mg/mL. Samples were stored in glass vials at 4° C. (solid circles), in glass vials at 37° C. (squares), in the MiniMed polypropylene reservoir at 37° C. (diamonds), and samples pumped with the MiniMed pump at 37° C. (triangles).

Drawing 7

Response of rats to subcutaneous injections of 120 μg/kg of GLP-1 in the preferred formulation (1 mg/mL GLP-1 dissolved in 10 mM sodium acetate, 5.07% D-mannitol, adjusted to pH 4.5). Values are the average of the response of 4 different animals.

Drawing 8

Total GRF detected in the plasma of a rat following intravenous administration of 20 μg of GRF in the preferred formulation (4 mg/mL GRF dissolved in 10 mM sodium acetate, 5.07% D-mannitol, adjusted to pH 4.7).

### DETAILED DESCRIPTION OF THE PREFERRED EMBODIMENTS

In accordance with the present invention, pharmaceutical formulations of a peptide, an acidic buffer and a diluent may be used for injection into a mammal. The peptide may have a molecular weight of between about 200 to 50,000 atomic mass units. According to a preferred embodiment, the peptide is a GLP-1 molecule, a PTH molecule, a GRF molecule, or a combination thereof. According to alternative embodiments, the peptide may be a derivative or an analog of GLP-1, PTH, GRF, or a combination thereof. According to a particularly preferred embodiment, the peptide is GLP-1(7-36)amide, PTH(1-34)OH, or GRF(1-44)amide.

The peptide concentration(s) (whether GLP-1, PTH, GRF, or combinations thereof) of the formulations are preferably in the range of about 25 μg to 5 mg per 1 mL of the combination of buffer and diluent.

GLP-1

According to a preferred embodiment of the present invention, the peptide is a glucagon-like peptide-1(7-36)amide. This molecule is a natural incretin hormone secreted from the L-cells of the ileum. It assists in the regulation of insulin secreatory rates and has a profound effect on glucose homeostasis. GLP-1 also acts systemically to suppress free fatty acids and to facilitate normalization of blood glucose levels through a large number of endocrine functions, including the control and expression of insulin from the pancreatic β-cells, and the suppression of glucagon. The term “GLP-1 molecule” as used in the context of the present invention includes glucagon-like peptides, analogs of glucagon-like peptide-1, and derivatives of glucagon-like peptide-1, that bind to glucagon-like peptide-1 receptor proteins.

Sequence of GLP-1(7-36)amide (Seq. 1):
His-Ala-Glu-Gly-Thr-Phe-Thr-Ser-Asp-Val-Sex-Ser-
Tyr-Leu-Glu-Gly-Gln-Ala-Ala-Lys-Glu-Phe-Ile-Ala-
Trp-Leu-Val-Lys-Gly-Arg-NH2.

According to an alternative embodiment of the present invention, an analog of GLP-1 may be used such as the GLP-1 derivatives:

Sequence of GLP-1(7-36)OH (Seq. 2):
His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-OH
Sequence of GLP-1(7-34)OH (Seq. 3):
His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-OH
Sequence of GLP-1(7-37)OH (Seq. 4)
His-Ala-Glu-Gly-Thr-Phe-Thr-Ser-Asp-Val-Ser-Ser-
Tyr-Leu-Glu-Gly-Gln-Ala-Ala-Lys-Glu-Phe-Ile-Ala-
Trp-Leu-Val-Lys-Gly-Arg-Gly-OH

Other GLP-1 analogs are known in the art. For example, U.S. Pat. No. 5,958,409 describes suitable GLP-1 analogs. According to other alternative embodiments, the peptide may be a GLP-1 derivative such as alkylated or acylated GLP-1 derivatives or other analogs. Analogs of GLP-1 that are homologous, including the exendins, such as exendin 3 and 4, and GLP-2, are also included in the invention. According to a particularly preferred embodiment, the GLP-1 molecule is GLP-1(7-36)amide, having the amino acid sequence Seq 1.

A factor that may play a role in the stability of the GLP-1 formulations is the concentration of the GLP-1 molecule. The solubility profile as a function of pH of GLP-1 is shown in Drawing 2. At pH values below about 5.0, the solubility of GLP-1 in 10 mM sodium acetate, 5.07% D-mannitol is generally above 1 mg/mL, allowing effective doses for s.c. and i.v. injections. The present inventors have determined that a GLP-1(7-36)amide concentration of about 1 mg/mL was stable in the inventive formulations at pH 4.5, for up to 6 months at 25° C. with ˜4% degradation. This stability was evidenced by the minimal amount of breakdown products (e.g., acid cleavage and beta shifts at aspartic acid) over time determined by HPLC methods. See Drawing 4. A particularly stable formulation includes about 0.1 to 4 mg/mL of a GLP-1 molecule.

Also included in “GLP-1 molecules” of the present invention are six peptides in Gila monster venoms that are homologous to GLP 1. Their sequences are compared to the sequence of GLP1 in the following table.

TABLE
Position 1
a. HAEGTFTSDVSSYLEGQAAKEFIAWLVKGR(NH₂)
b. HSDGTFTSDLSKQMEEEAVRLFIEWLKNGGPSSGAPPPS(NH₂)
c. DLSKQMEEEAVRLFIEWLKNGGPSSGAPPPS(NH₂)
d. HGEGTFTSDLSKQMEEEAVRLFIEWLKNGGPSSGAPPPS(NH₂)
e. HSDATFTAEYSKLLAKLALQKYLESILGSSTSPRPPSS
f. HSDATFTAEYSKLLAKLALQKYLESILGSSTSPRPPS
g. HSDAIFTEEYSKLLAKLALQKYLASILGSRTSPPP(NH₂)
h. HSDAIFTQQYSKLLAKLALQKYLASILGSRTSPPP(NH₂)
a = GLP-1(7-36)amide.
b = exendin 3.
c = exendin 4(9-39)(NH₂).
d = exendin 4.
e = helospectin I.
f = helospectin II.
g = helodermin.
h = Q8, Q9 helodermin.

The peptides c and h are derived from b and g, respectively. All 6 naturally occurring peptides (a, b, d, e, f, and g) are homologous in positions 1, 7, 11 and 18. GLP-1(7-36)amide and exendins 3 and 4 (a, b, and d) are further homologous in positions, 4, 5, 6, 8, 9, 15, 22, 23, 25, 26 and 29. In position 2, A, S and G are structurally similar. In position 3, residues D and E (Asp and Glu) are structurally similar. In positions 22 and 23, F (Phe) and I (Ile) are structurally similar to Y (Tyr) and L (Leu), respectively. Likewise, in position 26, L and I are structurally equivalent.

Thus, of the 30 residues of GLP1, exendins 3 and 4 are identical in 15 positions and equivalent in 5 additional positions. The only positions where major structural changes are evident are at residues 16, 17, 19, 21, 24, 27, 28 and 30. Exendins also have 9 extra residues at the carboxyl terminus.

PTH

According to another preferred embodiment of the present invention, the peptide is a PTH molecule. The term “PTH molecule” as used in the context of the present invention includes parathyroid hormones, analogs of parathyroid hormones, and derivatives of parathyroid hormones. PTHs are regulatory factors in the homeostatic control of calcium and phosphate metabolism. The principal sites of PTH activity are believed to be the skeleton, kidneys, and gastrointestinal tract.

Sequence of human PTH(1-34) (Seq. 5):
Ser-Val-Ser-Glu-Ile-Gln-Leu-Met-His-Asn-Leu-Gly-
Lys-His-Leu-Asn-Ser-Met-Glu-Arg-Val-Glu-Trp-Leu-
Arg-Lys-Lys-Leu-Gln-Asp-Val-His-Asn-Phe

According to an alternative embodiment of the present invention, an analog of PTH may be used. PTH analogs are known in the art. For example, U.S. Pat. No. 5,840,837 describes suitable PTH analogs. According to other alternative embodiments, the peptide may be a PTH derivative such as PTH(1-84), PTH(1-37) and C-terminal amidated derivatives of PTH or its derivatives, as examples. According to a particularly preferred embodiment, the peptide is PTH(1-34), a natural human PTH (Seq 5).

The present inventors have determined that a concentration of about 0.005 to 1.0 mg/mL of the PTH molecule was stable for 4 months at 4° C. in the inventive formulations. A particularly stable formulation includes about 0.02 to 0.10 mg/mL of PTH.

GRF

According to another preferred embodiment of the present invention, the peptide is GRF(1-44)amide (GRF). GRF is a peptide of 44 amino acids. GRF is one of a group of peptides secreted by the hypothalamus, and is believed to stimulate pituitary growth hormone release. GRF may be important in normal growth and development during childhood, and may mediate (together with somatostatin) the neuroregulation of GH secretion. GRF is an attractive molecule for the treatment of postmenopausal osteoporosis, and other indications because it is relatively small, and therefore can be effective when given by nasal insufflation using an appropriate vehicle.

The term “GRF molecule” as used in the context of the present invention includes growth hormone releasing factor, analogs of growth hormone releasing factor, and derivatives of growth hormone releasing factor, that bind to a growth hormone releasing factor receptor protein.

Sequence of GRF(1-44) amide (Seq. 6):
Tyr-Ala-Asp-Ala-Ile-Phe-Thr-Asn-Ser-Tyr-Arg-Lys-
Val-Leu-Gly-Glu-Leu-Ser-Ala-Arg-Lys-Leu-Leu-Gln-
Asp-Ile-Met-Ser-Arg-Gln-Glu-Gly-Glu-Ser-Asn-Gln-
Glu-Arg-Gly-Ala-Arg-Ala-Arg-Leu-NH₂.

According to an alternative embodiment of the present invention, an analog of GRF may be used. GRF analogs that have biological activity are known in the art and generally contain about 27 to about 44 amino acids, but such analogs may be somewhat less potent than GRF. For example, Kubiak et al, J. Med. Chem. 36, 888 (1993) describes suitable GRF analogs. Examples of GRF analogs that are included are GRF(1-44)-OH, GRF(1-40)-OH, GRF(1-40)-NH₂, GRF(1-32)-NH₂, GRF(1-39)-NH₂, GRF(1-40)-Phe-NH₂, GRF(1-40)-Phe-OH, GRF(1-40)-Phe-Gln-NH₂, GRF(1-29)-NH₂, and GRF(1-27)-NH₂, and combinations thereof. According to other alternative embodiments, the peptide may be a GRF derivative such as detailed by Kubiak et al. above. According to a particularly preferred embodiment, the peptide is GRF (1-44) amide having the amino acid sequence of Seq. 6. A particularly stable formulation for GRF includes about 1.0 to 10.0 mg/mL of GW.

Buffer

The buffer of the formulations should have a pH that is slightly acidic. Without intending to be limited by any particular theory, it is known to those skilled in the art that acidic conditions increase the stability of the amide bond of the peptide. Acidic conditions are provided by a generally weak acid. An acid is a generally weak acid if it has an acid dissociation constant value of greater than about 1×10⁻⁵, or greater than 1×10⁻⁵, i.e., a pKa<about 5, or a pKa<5. Such acids may include propionic, succinic, malic acids, and combinations thereof. According to a particularly preferred embodiment, the acid is acetic acid. According to an alternative embodiment, the acid may have an acid dissociation constant value greater than about 1×10⁻⁵, or greater than 1×10⁻⁵, (such as proprionic or lactic acids). The buffer may have buffering capabilities and may be selected from the group consisting of acetates, borates, phosphates, phthalates, carbonates, and combinations thereof. In one preferred embodiment, the buffer is included in a solution including the peptide and excipient to establish a pH in the range of about 3.0 to about 5.0. It is well known in the art that pH can be adjusted to a desired range using well known reagents, such as weak acids, as described herein, and strong bases, such as sodium or potassium hydroxide. In another preferred embodiment, the pH of the buffer is in the range of 3.0 to 5.0. In more preferred embodiments, the pH of the buffer is in the range of about 4.0 to about 5.0 or 4.0 to 5.0. In more particularly preferred embodiments, the pH of the buffer is in the range of about 4.5 to about 5.0 or 4.5 to 5.0. In a most preferred embodiment, the pH of the buffer is in the range of about 4.5 to about 4.7 or 4.5 to 4.7. In yet other most preferred embodiments, the pH of the buffer is 4.5, 4.6 or 4.7. The buffer preferably has a molarity of between about 1 mM and 20 mM, more preferably in the range of between about 5 and 10 mM.

Isotonic Excipient

The excipient assists in rendering the formulations approximately isotonic with body fluid (depending on the mode of administration). The concentration of the excipient is selected in accordance with the known concentration of a tonicity modifier in a peptide formulation. Preferred excipients include saccharides, such as lactose or D-trehalose having a chemical composition of C₁₂H₂₂O₁₁. A particularly preferred excipient (also sometimes referred to as a “diluent” in this context) in the present invention is D-mannitol, having a chemical composition of C₆H₁₄O₆. Other preferred excipients include alcohols having a C₁ to C₁₂ chain. According to alternative embodiments, the excipient may include, but is not limited to, saline, buffered saline, dextrose, water, glycerol, ethanol, lactose, D-mannitol, arginine, other amino acids, and combinations thereof.

Novel Formulations

The compositions of the present invention are novel peptide formulations that are well-suited for clinical therapeutic administration, because (1) they may be sterilized, (2) may have controlled tonicity, (3) may be pH-adjusted, and (4) are compatible with administration in a variety of ways. An unexpected property of embodiments of the inventive formulations is that despite their relatively low pH, they produce little or no adverse side effects in patients, when administered parenterally. Moreover, in studies with animals and humans, both subcutaneous and intravenous injections of the peptides produce biological responses indicative of their function.

The inventors of the present invention have found that an acceptable solubility of the peptide in the formulations is possible at a low pH range. According to particularly preferred embodiments, at least about 2 mg of GLP-1, at least about 4 mg PTH, or at least about 10 mg of GRF peptide is soluble in about 1 mL of the buffer and the excipient combined, when the formulation has a pH in the range of about 4.0 to 5.0, or 4.0 to 5.0. These inventive formulations preferably are substantially free of agents such as detergents, solvents, or other adjuvants or excipients, that would be required for adequate peptide solubility at higher pH values.

In preferred embodiments, the inventive formulations comprise acetic acid, D-mannitol, and a molecule selected from the group consisting of a GLP-1 molecule, a GRF molecule, and a PTH molecule, and have a pH of about 4.5 to about 4.7, or 4.5 to 4.7. In other preferred embodiments, the inventive formulations consist essentially of acetic acid, D-mannitol, and a molecule selected from the group consisting of a GLP-1 molecule, a GRF molecule, and a PTH molecule and have a pH of about 4.5 to about 4.7, or 4.5 to 4.7. In other preferred embodiments, the inventive formulations consist of acetic acid, D-mannitol, and a molecule selected from the group consisting of a GLP-1 molecule, a GRF molecule or a PTH molecule and have a pH of about 4.5 to about 4.7 or 4.5 to 4.7. In still other preferred embodiments, the inventive formulations have a pH of about 4.5, a pH of about 4.6, a pH of about 4.7, a pH of 4.5, a pH of 4.6, or a pH of 4.7.

A pH range of between about 4.0 to 5.0 has not presented problems with precipitation at the site of injection, even though the peptide may be rather insoluble at physiological pH. Test results show that blood glucose falls to euglycemic levels within 10 minutes of injection of GLP-1 in a human subject, which indicates that generally none of the peptide precipitated at the site of injection. When GLP-1 or GRF formulations were injected subcutaneously in the amount of about 1 mL into humans, they produced no apparent discomfort at the injection site and produced a rapid response, as assessed by the level of peptide drug appearing in the blood.

The formulations of the present invention are surprisingly stable even when injected in a human subject. The biological half-life of peptide molecules is quite short. For example, the biological half-life of GLP-1(7-37) in blood is 3 to 5 minutes, according to U.S. Pat. No. 5,118,666. Without intending to be limited by any particular theory, it is believed that the effectiveness of these inventive formulations in part results from a combination of the identity and pH of the buffer and the stabilizing effect of the excipient (e.g., D-mannitol). The inventors of the present invention have developed HPLC methods capable of quantifying the degree of degradation of the peptide (See Drawing 1).

The formulations of the present invention comprising GLP-1 were used in human patients in clinical trials and caused few adverse effects. In excess of 10,000 vials of such formulations have been stable for at least a period of 9 months at −20° C., 4° C., and 25° C. The formulations of the present invention where the peptide is GRF or PTH also exhibit comparable stability (See Drawings 3, 5).

Referring to Table 1, a formulation of 1 mg/mL GLP-1 in 10 mM acetate, 5.07% (w/v) D-mannitol, and pH 4.5, showed a stability of at least 98% over 28 days at 25° C.; at least 92% over 28 days at 37° C., and at least 66% over 28 days at 50° C. Moreover, this GLP-1 formulation showed no change in purity when stored for one month at 4° C. or −20° C. An additional stability study showed at least 90% stability of GLP-1 in this formulation over 18 months at 4° C. and 6 months at 25° C.

Formulations of PTH(1-34) at 0.1, 1.0 and 10.0 mg/mL, pH 4.7, 5.07% D-mannitol, 10 mM acetate were highly stable, at least about 98% over 14 days at temperatures from −20° C. to 25° C. At 50 μg/mL in the same formulation, PTH(1-34) was shown to be at least 90% stable for more than 6 months at −20° C. and 5° C., and for three months at 25° C.

GRF formulations at 4, 8, and 10 mg/mL, pH 4.7, 5.07% D-mannitol, 10 mM acetate, at temperatures from −20° C. to 4° C. showed a stability of at least 98% over 14 days, at least 96% at 25° C. and 63% at 50° C. Additional formulations tested for extended periods of time showed stability of at least 90% for 12 months at 4° C., and 4-6 weeks at 25° C.

Therefore, the formulations of the present invention include peptides that are very stable and storable, probably for years at −20° C. Also, their decomposition at higher temperatures yields fragments that have been identified and are predictable. There has been no detectable dimerization or aggregation of these formulations.

Preparation of Peptides

The peptides of the present invention may be prepared by methods as are generally known in the art of peptide preparation. For example, the peptides can be prepared by solid-state chemical peptide synthesis or by conventional recombinant techniques. The term “recombinant” means that a desired peptide or protein is derived from recombinant (e.g., microbial or mammalian) expression systems. The basic steps and techniques in recombinant production are well-known to the ordinarily-skilled artisan in recombinant DNA technology and include (1) isolating a natural DNA sequence encoding a peptide molecule of the present invention or constructing a synthetic or semi-synthetic DNA coding sequence for a peptide molecule; (2) placing the coding sequence into an expression vector in a manner suitable for expressing proteins either alone or as a fusion protein; (3) transforming an appropriate eukaryotic or prokaryotic host cell with the expression vector; (4) culturing the transformed host cell under conditions that will permit expression of a peptide molecule; and (5) recovering and purifying the recombinantly produced peptide molecule. The peptides can be recovered and purified from recombinant cell cultures by methods including, but not limited to, ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. High performance liquid chromatography (HPLC) can be employed for final purification steps.

Therapeutic Methods and Administration

The formulations of the present invention have a variety of uses for treating disease and illness in mammals. The skilled artisan will recognize that the present inventive formulations can be used for any disease or condition that requires parenteral administration of a GLP-1 molecule, a GRF molecule, or a PTH molecule. The formulations including GLP-1 may be useful for treating diabetes, excess appetite, and obesity. The formulations including PTH may be useful for treating bone growth deficiency and osteoporosis. The formulations including GRF may be useful for treating osteoporosis and wasting; patients who have been injected with formulations of the present invention have had minimal or no irritation at all upon injection and have experienced a growth hormone response, which indicates that the peptide gets into the circulation.

The formulations of the present invention are preferably administered in unit dosage form. In such form, the formulations are subdivided into unit doses containing appropriate quantities of the peptide. The unit dose can be a packaged preparation, the package containing discrete quantities of peptide, such as liquid containing solubilized peptide in vials or ampoules, packeted tablets, capsules, and powders in vials or ampoules. The determination of the proper dose for a particular situation is within the skill of the art. In general, treatment is initiated with smaller doses, which are less than the optimum dose of the preparation. Thereafter, the dose is increased by small increments until the optimum effect under the circumstances is reached. For convenience, the total daily dose may be divided and administered in portions during the day, if desired.

A typical unit dose of a formulation including GLP-1 is about 0.1 to 2 mg or 0.1 to 2 mg, about 10 to 50 μg for a formulation including PTH, and about 1 to 8 mg or 1 to 8 mg for a formulation including GRF, though doses above and below these amounts may have application. According to a particularly preferred embodiment, the doses are liquid formulations of about 1 mg/mL of GLP-1, about 50 μg/mL of PTH, or 50 μg/mL, and about 1 to 4 mg/mL of GRF or 1 to 4 mg/mL, each dose is made up in standard 3 mL vials and filled at a commercial facility (e.g., SP Pharmaceuticals in New Mexico).

The formulations of the present invention are primarily intended for administration to a human subject, but may also be administered to other mammalian subjects, such as dogs and cats (e.g., for veterinary purposes). The formulations can also be preferably administered for continuous subcutaneous delivery using, for example, a MiniMed® programmable medication infusion pump commercially available from Pacesetter Systems, Inc., of California. In vitro and in vivo studies show minimal adsorption of the formulations to components of the MiniMed pump. Further, the formulations in the preferred embodiment can be diluted up to 40-fold with isotonic saline and delivered by pump, such as the Harvard pump, Harvard Apparatus, MA, without loss of biological activity nor adsorption of peptide.

Referring to Drawing 6, a study of the stability of the GLP-1 formulation stored at 4° C. and 37° C. in glass vials and in the polyproplyene reservior of the MiniMed pump system as well as the stability of the formulation being pumped for 6 days show a high degree of stability, indicating usefulness as a delivery method, with neither loss of material nor degradation of the peptide over that time period.

Extensive experience with the preferred formulations of GLP-1 and GRF in human subjects with both intravenous and subcutaneous delivery has indicated good delivery of the peptide with no significant complications; little inflammation or discomfort is reported by patients. According to alternative embodiments, the formulations may be delivered by other means, including subcutaneous or micropressure injection, external or implant pump, depot injection, and other prolonged-application dispensing devices. Alternatively, in other embodiments, a syringe can be used that comprises an inventive formulation of the present application. Such a syringe, can be used for self-administration of a GLP-1 molecule. Such syringes are well known in the art. See, e.g., U.S. Pat. Nos. 5,980,491 and 5,984,900.

According to an alternative embodiment of the present invention, the formulations may be sterile. The term “sterile” as used in the context of the present invention means aseptic or substantially free of microorganisms. The formulations may be made sterile by the destruction or removal of substantially all microorganisms by a variety of methods known in the art including, but not limited to, physical methods (e.g., heat, sound, light, radiation, adsorption, filtration) and chemical methods (e.g., antiseptics).

The present inventive formulations may be embodied in other specific forms without departing from its spirit or its central characteristics. The described embodiments are to be considered in all respects only as illustrative and not restrictive. The scope of the invention is, therefore, indicated by the appended claims, rather than by the foregoing description. All changes that come within the meaning and range of equivalency of the claims are to be embraced within their scope. For example the formulations of the present invention may include a pharmaceutically acceptable preservative, a tonicity modifier, an adjuvant or auxiliary drug to assist the action of the peptide, an excipient or an inert carrier for the peptide, a detergent such as TWEEN 80, or a solvent to increase the solubility of the peptide.

The following examples and preparations are provided merely to further illustrate the preparation, stability and effectiveness of the formulations of the invention. The scope of the invention is not limited to the following examples.

### EXAMPLES

### Example 1

GLP-1, PTH, and GRF, as their chloride salts, were dissolved in the formulation at the pH values indicated in Table 1, vialed in 1 mL tubing glass vials and stoppered with Helvoet Omniflex stoppers and metal crimp seals (SP Pharmaceuticals, NM). The vials were stored at the indicated temperatures for the indicated times. Samples were removed and assayed for the loss of parent peptide by HPLC, using a reversed phase C18 (1×15 cm) analytical column. Samples (10 μl) were injected directly and resolved with a gradient of acetonitrile in water, in the presence of 0.1% trifluoroacetic acid. Percent peptide remaining at the times indicated was calculated as the area of the intact peptide divided by the total area of the intact peptide plus that of the decomposition products times 100.

TABLE 1
Stability of GLP-1, PTH, and GRF in the preferred
formulation as a function of time and temperature.

| | Concen- | Percent peptide remaining | | | |
|---|---|---|---|---|---|
| Formulation | tration | 4° C. | 25° C. | 37° C. | 50° C. |
| GLP-1; 10 | 1 mg/mL, | 99 | 98 | 92 | 66 |
| mM acetate, | 1 month | | | | |
| 5.07% (w/v) | | | | | |
| D-mannitol, | | | | | |
| pH 4.5 | | | | | |
| GRF; 10 | 4 mg/mL, | 98 | 96 | ND | 63 |
| mM acetate, | 14 days | | | | |
| 5.07% (w/v) | | | | | |
| D-mannitol, | | | | | |
| pH 4.7 | | | | | |
| GRF; 10 | 8 mg/mL, | 98 | 96 | ND | 63 |
| mM acetate, | 14 days | | | | |
| 5.07% (w/v) | | | | | |
| D-mannitol, | | | | | |
| pH 4.7 | | | | | |
| GRF; 10 | 10 mg/mL, | 98 | 96 | ND | 63 |
| mM acetate, | 14 days | | | | |
| 5.07% (w/v) | | | | | |
| D-mannitol, | | | | | |
| pH 4.7 | | | | | |
| PTH; 10 | 0.1 mg/mL, | 98 | 98 | ND | 75 |
| mM acetate, | 14 days | | | | |
| 5.07% (w/v) | | | | | |
| D-mannitol, | | | | | |
| pH 4.7 | | | | | |
| PTH; 10 | 1 mg/mL, | 98 | 96 | ND | 74 |
| mM acetate, | 14 days | | | | |
| 5.07% (w/v) | | | | | |
| D-mannitol, | | | | | |
| pH 4.7 | | | | | |
| PTH; 10 | 10 mg/mL, | 98 | 97 | ND | 76 |
| mM acetate, | 14 days | | | | |
| 5.07% (w/v) | | | | | |
| D-mannitol, | | | | | |
| pH 4.7 | | | | | |

### Example 2

The stability of GRF(1-44)amide was investigated in various formulations. GRF(1-44)amide was formulated as listed in Table 2 and the purity after 7 days at various temperatures was measured using a Beckman HPLC commercially available from Beckman Instruments, CA, using a reversed phase C 18 analytical column with a gradient of increasing acetonitrile in water, in the presence of 0.1% trifluoroacetic acid.

TABLE 2
GRF solubility/stability in formulations after storage at 4° C.,
25° C., and 50° C. for 7 days at 4 mg/mL.

| | Formulation | 4° C. | 25° C. | 50° C. |
|---|---|---|---|---|
| A. | Water, pH 2.9 | 99% | 99% | 63% |
| B. | 10 mM acetate, 10% (w/v) lactose, | 99 | 98 | 79 |
| | pH 4.8 | | | |
| C. | 10 mM bicarbonate, 10% (w/v) lactose, | 99 | 74 | 34 |
| | pH 7.5 | | | |
| D. | unbuffered, 10% (w/v) lactose, pH 2.9 | 99 | 96 | 59 |
| E. | 10 mM acetate, 5.07% (w/v) D- | 99 | 99 | 89 |
| | mannitol, pH 4.7 | | | |
| F. | 10 mM bicarbonate, 5.07% (w/v) D- | 99 | 93 | 42 |
| | mannitol, pH 7.7 | | | |
| G. | unbuffered, 5.07% (w/v) D-mannitol, | 99 | 97 | 63 |
| | pH 2.9 | | | |
| H. | 10 mM acetate, 2% (w/v) D-trehalose, | 99 | 99 | 88 |
| | pH 4.7 | | | |
| I. | 10 mM bicarbonate, 2% (w/v) D- | 98 | 92 | 39 |
| | trehalose, pH 7.7 | | | |
| J. | unbuffered, 3% (w/v) D-trehalose, | 99 | 97 | 63 |
| | pH 2.9 | | | |

The data from Table 2 indicate that bicarbonate (formulations C, F, I) appears to accelerate degradation of the peptide. Lactose (formulations B, C, D) appears to be inferior to D-mannitol (formulations E, F, G) in preventing degradation of the peptide under any condition, and D-trehalose (formulations H, I, J) appears to stabilize the peptide almost as well as D-mannitol. The major breakdown products in the acetate formulations (formulations B, E, H) were acid cleavage and beta shifts at aspartic acid. The major breakdown products in the bicarbonate (formulations C, F, I) were unknown.

The unique properties of the preferred formulation, particularly with GLP-1, is illustrated in Table 3, where it is shown that numerous attempts to prepare 1 mg/mL isotonic formulations with GLP-1 failed, largely because of particulate formation, as evidenced by light scattering, and precipitate/gel formation. The clearly evident light scattering observed, even when a standard solubilizing excipient such as Tween 80 was used, makes such formulations suboptimal and impractical.

TABLE 3

| Formulation | | Result |
|---|---|---|
| A. | 10 mM sodium acetate, 0.9% (w/v) NaCl, | Scatters at 37° C. |
| | pH 4.0 | |
| B. | 10 Mm sodium acetate, 0.9% (w/v) NaCl, | Scatters at 37° C. |
| | pH 4.5 | |
| C. | Formulation B with 0.00004% Tween 80 | Scatters at 37° C. |
| D. | 10 mM sodium lactate, 0.9% (w/v) NaCl, | Scatters at 37° C. |
| | pH 4.0 | |
| E. | 10 mM sodium lactate, 0.9% (w/v) NaCl, | Scatters at 37° C. |
| | pH 4.5 | |
| F. | Formulation E with 0.00004% Tween 80 | Scatters at 37° C. |
| | | and 25° C. |
| G. | 10 mM phosphate, 0.9% (w/v) NaCl, | Precipitate at 25° C. |
| | pH 8.0 | |
| H. | 10 mM phosphate, 0.9% (w/v) NaCl, | Precipitate at 25° C. |
| | pH 8.5 | |
| I. | Formulation H with 0.00004% Tween 80 | Clear |

### Example 3

### Long-Term Stability in the Preferred Embodiment

GLP-1, GRF, and PTH were formulated at SP Pharmaceuticals under cGMP guidelines in 10 mM acetate, 5.07% D-mannitol in 3 mL glass vials with Helvoet stoppers and metal seals. The vials containing 1 mL of formulated drug were put into thermostatted chambers and assayed for % peptide remaining as a function of time after storage at different temperatures. Bioactivity of the formulations at the time points was also measured.

Drawings 3, 4, and 5 show results that demonstrate that the formulations are highly stable for at least 9 months at −20° C. and 4° C. as assessed by decomposition (measured by HPLC) and/or bioactivity. GLP-1 formulation stability data is presented in Drawing 4 and PTH formulation stability data is shown in Drawing 5.

The bioactivity of PTH was determined by the chick hypercalcemia assay of Parsons et al., Endocrinology 92, 454 (1973). GLP-I bioactivity was measured using the transformed human kidney fetal kidney 293 cell line containing a constitutively expressed receptor for GLP-1. GRF activity was assessed similarly using a cell line containing an expressed GRF receptor and monitoring the response of cell to GRF by the CAMP-responsive secreted alkaline phosphatase reporter system.

### Example 4

The solubility of GLP-1 as a function of pH was examined and shown to have the pH-solubility profile shown in Drawing 2. This hormone has maximal solubility under acidic conditions (pH<4) but at pH values of 5 and above the solubility is less than 1 mg/mL. At pH 4.6 the solubility is about 12 mg/mL.

### Example 5

To illustrate that the preferred formulations deliver peptide rapidly and effectively to animals, rats were injected subcutaneously with GLP-1 in the preferred formulation and the plasma was assayed for GLP-1 by conventional immunoassay for total GLP-1 as a function of time. The injected GLP-1 caused a rapid increase in plasma levels, shown in Drawing 7, indicating rapid and significant delivery of the peptide. Similarly, Drawing 8 shows that when a rat is given an intravenous bolus of 20 μg of GRF formulated in 10 mM sodium acetate, 5.07% D-mannitol, pH 4.7, the peptide rapidly appears in the blood plasma.

### Example 6

GLP-1 formulated and delivered subcutaneously continuously over 24 hours produced plasma concentrations of GLP-1 about 6-fold above basal levels in man. Thus, GLP-1 dissolved at 1 mg/mL in 5.07% D-mannitol and 10 mM sodium acetate at pH 4.5 was placed in a MiniMed 507 infusion pump and delivered subcutaneously to a human subject at a rate of 2.4 pmol/kg/min for 24 hours. The mean (n=7) basal GLP-1 concentration in plasma prior to infusion measured by radioimmunoassay was 24.7 pM and that during infusion was 147 pM, illustrating that continuous sc infusion of the formulation leads to substantial increases in plasma GLP-1.

What is claimed is:

1. A pharmaceutical composition comprising:
a GRF molecule;
an acid having a dissociation constant value of greater than 1×10⁻⁵; and
an excipient;
wherein the pH of said composition is between about 3.0 and 5.0 and wherein the excipient includes at least D-mannitol.

2. The composition according to claim 1, wherein said acid comprises acetic acid.

3. The composition according to claim 1, wherein said excipient is D-mannitol.

4. The composition according to claim 1 wherein said acid is acetic acid and said excipient is D-mannitol.

5. The composition according to claim 1, wherein said composition comprises GRF(1-44)amide.

6. The composition of claim 1, wherein said composition is in unit dosage form.

7. The composition of claim 1, wherein said composition is sterile.

8. A system for administering a pharmaceutical composition comprising:
an infusion pump for administering a unit dose of the composition according to claim 1.

9. A system of claim 8, wherein said composition is diluted up to about 40-fold with isotonic saline prior to administration.

10. A method for the treatment of a disease or condition in a mammal, which may be treated or prevented by growth hormone, comprising administering to the mammal a pharmaceutically effective amount of a composition according to claim 1.

11. The method of claim 10, wherein the disease or condition osteoporosis.

12. The method of claim 10, wherein said composition is administered to said mammal by a method selected from the group consisting of intravenous, subcutaneous, continuous, intermittent, parenteral, and combinations thereof.
